(19) United States
(12) Patent Application Publication
(10) Pub. No.: US 2025/0161231 A1
(43) Pub. Date: May 22, 2025

(54) LUTEIN FOR USE IN INHIBITING MAST CELL DEGRANULATION

(71) Applicants: N.V. NUTRICIA, Zoetermeer (NL); NUTRICIA EARLY LIFE NUTRITION (SHANGHAI) CO., LTD., Shanghai (CN)

(72) Inventors: Catharina Theresia KNIPPING, Utrecht (NL); Johan GARSSEN, Utrecht (NL); Thomas LUDWIG, Utrecht (NL); Nicole BUURMAN, Utrecht (NL); Haofang JIN, Utrecht (NL)

(73) Assignees: N.V. NUTRICIA, Zoetermeer (NL); NUTRICIA EARLY LIFE NUTRITION (SHANGHAI) CO., LTD., Shanghai (CN)

(21) Appl. No.: 18/969,363

(22) Filed: Dec. 5, 2024

Related U.S. Application Data
(63) Continuation of application No. PCT/CN2023/098011, filed on Jun. 2, 2023.

(30) Foreign Application Priority Data
Jun. 5, 2022 (WO) PCT/CN2022/097145

(51) Int. Cl. A61K 31/047; A23L 33/00; A61K 31/231; A61K 31/7016; A61K 31/702; A61K 38/17

(52) U.S. Cl. CPC A61K 31/047; A23L 33/40; A61K 31/231; A61K 31/7016; A61K 31/702; A61K 38/1709

(57) ABSTRACT

The present invention relates to nutritional compositions comprising lutein for inhibiting mast cell degranulation, thereby preventing or treating symptoms or disorders associated with mast cell degranulation. Preferably, the compositions comprise lutein and non-digestible carbohydrates, such as those selected from prebiotic oligosaccharides, human oligosaccharides and combinations thereof.

### FIELD OF THE INVENTION

The present invention relates to nutritional compositions comprising lutein for inhibiting mast cell degranulation, thereby preventing or treating symptoms or disorders associated with mast cell degranulation.

### BACKGROUND OF THE INVENTION

Mast cells have a relevant function in the innate and adaptive immune system. Mast cells are found in a wide variety of tissues and are able to recognize allergens by binding to IgE bound to their receptors on their membranes. Mast cells are thus critical constituents in allergic reactions and inflammation.

Hundreds of secretory granules are present in each mast cell. When triggered by specific or non-specific mechanisms such as allergic reactions involving immunoglobulin E (IgE) and antigens, each of these granules secrete the content thereof which include biological compounds directly involved e.g. in the inflammatory cascade. Moreover, the compounds released by the mast cells following degranulation are known to cause many biological responses which are part of the overall response of the body to invasion by infective organisms, allergens or other stressful stimuli. For instance, when histamine is released by mast cell degranulation, it binds to specific receptors on the surface of endothelial cells on vessels, neurons or other tissues causing vasodilation and chemoattraction and permitting lymphocytes to leave the blood circulation and enter the tissues, where they cause additional mast cell degranulation and other responses. The process of degranulation thus continues and worsens.

The existence of “mast cell activation disorders” started being reported in the literature in the 1980's, apparently associated with sudden synchronous mediator release in the absence of evidence of mast cell proliferation (Akin et al. ‘Mast cell activation syndrome: proposed diagnostic criteria. J Allergy Clin Immunol. 2010 December; 126(6):1099-104.e4.). Non-allergic triggers are certain polysaccharides or monosaccharide-containing lectins, such as dextran, the neurotransmitter acetylcholine, various neuropeptides, as well as viruses, bacterial toxins, drugs (such as aspirin, morphine and curare), contrast media used in radiology, extreme heat, cold, solar radiation, hyperosmotic media and pressure.

Degranulation of mast cells and basophils are also an essential step in the process of inflammation induction and are part of the effector phase of the allergic reaction. After crosslinking, the mast cell degranulates and thereby rapidly releases inflammatory mediators including histamine, eicosanoids, proteolytic enzymes, cytokines, and chemokines from the granules which amplifies the inflammatory response. The degranulation of mast cells and the accumulation of eosinophils in the sites of inflammation are considered to result from the unbalanced overactivation of Th2 cells and consequently the Th2-mediated overproduction of IgE (Abbas et al., 1991, Nature 383:787-93; Vercelli, 2001, Curr Opin Allergy Clin Immunol 2001, 1:61-5). Suppression of degranulation is therefore believed to be effective as a method for the treatment of such diseases.

U.S. Pat. No. 6,689,748B1 describes a method of treating mast cell-induced diseases such as allergies, interstitial cystitis, inflammatory bowel disease, irritable bowel syndrome, and certain hyperproliferative diseases such as systemic mastocytosis, the method comprising the administration to said mammal of a pharmaceutically effective amount of a proteoglycan with mast cell secretion inhibitory activity, said proteoglycan comprising a chondroitin sulphate, alone or together with one or more synergistic adjuvants.

Carotenoids are known to have antioxidant functions and more research has shown some immunomodulatory effect of carotenoids in relation to cardiovascular diseases and cancer. There are more than 600 types of carotenoids which can be broadly classified in carotenes and xanthophylls. Lutein is a xanthophyll and is a natural part of the human diet found in orange-yellow fruits and flowers, and in leafy vegetables. The main benefits of including lutein in the diet as of an early age have been reported to be associated with neuro-development, especially visual acuity and development of the eye as part of the central nervous system.

US2003/0228392 describes infant formula compositions containing lutein and zeaxanthin preferably in an amount of 6 to 230 μg/L.

US2007/0098849 discloses infant formulas comprising docosahexaenoic acid (DHA) and lutein to promote retinal health and vision development in infants. It describes a weight ratio of lutein:DHA from about 1:2 to about 10:1.

Schmutzler et al. (Effect of Beta-Carotene on Histamine Release from Human Mast Cells and Monocytes, Int Arch Allergy Immunol 113, 335-336, 1997) describe a dose-dependent inhibition by beta-carotene of histamine release from human mast cells.

Manabe et al. (Suppressive effects of carotenoids on the antigen-induced degranulation in RBL-2H3 Rat Basophilic Leukemia cells. J. Oleo Sci. 63 (3) 291-294. 2014) describe an in vitro study to evaluate the anti-degranulation effects of carotenoids using RBL-2H3 rat basophilic leukemia cell line as a mast cell model.

### SUMMARY OF THE INVENTION

The present inventors have surprisingly found that a nutritional composition comprising lutein can specifically suppress mast cells degranulation, thereby preventing and/or treating the symptoms or disorders associated with mast cell degranulation.

Accordingly, in a first aspect, the invention relates to a nutritional composition comprising lutein for use in the prevention or treatment of a mast cell degranulation associated disorder in a human subject.

In a second aspect, the invention relates a composition comprising lutein for use in inhibiting allergic symptoms in a human subject, wherein the allergic symptoms are caused by mast cell degranulation.

Additionally, the inventors have surprisingly found that the combination of lutein and short chain fatty acids (i.e., the fermentation products of non-digestible carbohydrates) can synergistically inhibit mast cell degranulation, therefore providing an useful therapeutic approach to treat or prevent conditions and symptoms associated with mast cell degranulation.

In a further aspect, the invention relates to a nutritional composition comprising carbohydrates, proteins and lipids, wherein the composition is a powder composition comprising, per 100 g:
a. an energy content of at least 400 kcal;
b. 60-430 μg lutein;
c. 3.5-8 g non-digestible carbohydrates comprising fructo-oligosaccharides and galacto-oligosaccharides;
d. 45-70 g digestible carbohydrates, preferably lactose;
the composition further comprising at least one of:
e. 25-80 mg casein phosphopeptides; or
f. 2-8 g 1,3-Dioleoyl-2-palmitoyl glycerol.

### DRAWINGS

FIG. 1: RBL degranulation assay for inhibitory effect of IgE-mediated mast cell degranulation of lutein.

FIGS. 2(a)-(c): RBL degranulation assay for inhibitory effect of IgE-mediated mast cell degranulation using mixtures comprising lutein and short chain fatty acids.

FIG. 3: RBL degranulation assay for inhibitory effect of non-IgE-mediated mast cell degranulation of lutein.

FIGS. 4(a)-(c): RBL degranulation assay for inhibitory effect of non-IgE-mediated mast cell degranulation of lutein and short chain fatty acids.

### DETAILED DESCRIPTION OF THE INVENTION

In a first aspect, the invention relates to a nutritional composition comprising lutein for use in the prevention or treatment of a mast cell degranulation associated disorder in a human subject.

In some jurisdictions, the present invention is best described as a (therapeutic) method of preventing or treating a mast cell degranulation associated disorder in a human subject, the method comprising administering to the human subject a composition comprising lutein.

The invention may also be described as the use of lutein in the manufacture of a nutritional composition for preventing or treating a mast cell degranulation associated disorder in a human subject.

The invention also refers to a nutritional composition comprising lutein for use in inhibiting allergic symptoms in a human subject, wherein the allergic symptoms are caused by mast cell degranulation. Preferably, the compositions are for avoiding allergic symptoms, preferably during introduction or reintroduction of allergenic foods such as cow's milk.

In some jurisdictions, the invention is defined as a (therapeutic) method of inhibiting allergic symptoms in a human subject, the allergic symptoms being caused by mast cell activation, wherein the method comprises administering to the human subject a composition comprising lutein. The invention may also be defined as the use of lutein in the manufacture of a nutritional composition for inhibiting allergic symptoms in a human subject, wherein the allergic symptoms are caused by mast cell activation.

The lutein is present in the composition in a therapeutically efficient amount.

Prevention, as used herein, refers to reduction of the risk of developing symptoms or conditions associated with mast cell degranulation in a human subject, particularly a human subject at risk of developing such symptoms or conditions. The treatments encompassed by the present invention include alleviation of symptoms or attenuation of manifestation of the disorders associated with mast cell degranulation.

“Mast cell degranulation”, as used herein, refers to the release of any or all mediators from any or all secretory granules of mast cells, whether in parallel, sequentially, differentially or selectively. Relevant examples of such responses and symptoms associated with mast cell degranulation include vasodilation and recruitment of inflammatory cells (e.g. leukocytes) from the circulation, tearing, nasal secretions, bronchoconstriction, itching of the skin, diarrhea or bladder pain.

“Mast cell degranulation associated disorders” are hereby characterized by exaggerated release of mast cell granule contents. It can be classified as primary (e.g. mastocytosis) and secondary mast cell degranulation disorders. Secondary mast cell degranulation disorders include allergies, mast cell dysfunction in chronic atopic and inflammatory disorders, such as helminth infections, inflammatory bowel disease, asthma, retinopathy (e.g. retinopathy of prematurity), hypoxic-ischemic encephalopathy, bronchopulmonary dysplasia, sudden infant death syndrome, atherosclerotic vascular disease, vascular aneurysms (e.g. brain, aorta), and neovascularization of tumours. Preferably, the mast cell degranulation associated disorder is a secondary mast cell degranulation disorder.

Preferably, the disorders associated with mast cell degranulation are allergies, including IgE-mediated and/or non-IgE-mediated allergies, and allergic inflammation. In a preferred embodiment, the disorder is allergy. According to one embodiment, the allergy is IgE-mediated allergy. According to another embodiment, the allergy is non-IgE-mediated allergy. In a particular embodiment, the human subject may suffer from both IgE-mediated and non-IgE-mediated allergy.

In a preferred embodiment, the disorder is allergy, preferably food allergy. Food allergies may be divided into 2 types: IgE-mediated and non-IgE-mediated. Non-IgE mediated food allergy reactions do not appear immediately after the ingestion of the food and usually relate to reactions in the gastrointestinal tract such as vomiting, bloating and diarrhoea. In contrast, IgE-mediated food allergy reactions usually occur within minutes of ingestion and include hives, redness of the skin, vomiting and in more severe reactions, anaphylaxis. Accordingly, in the present invention, the food allergy may be IgE-mediated and/or non-IgE mediated food allergy.

Food allergies in the context of the invention include allergies caused by the exposure of the human subject to allergens present in milk, egg, soy, wheat, peanut, tree nuts, fish and/or shellfish. Preferably, the allergy is egg and/or milk allergy, more preferably milk allergy.

Likewise, symptoms of disorders associated with mast cell degranulation include allergic reactions such as allergic inflammation. Non-limiting examples of symptoms particularly alleviated by the composition of the present invention comprise tingling or itching in the mouth, difficulty swallowing, hives, itching or eczema, swelling of the lips, face, mouth, tongue and, or throat or other parts of the body, wheezing or shortness of breath, nasal congestion or trouble breathing, abdominal pain, diarrhea, nausea or vomiting, hay fever-like symptoms, such as sneezing or itchy eyes.

### Lutein

As used herein, lutein is in the form of free xanthophylls, xanthophyll esters or other chemical forms of lutein. Lutein may be obtained or isolated by any method recognized by those skilled in the art. For example, lutein may be obtained by extraction from marigolds or other xanthophylls-rich sources, chemical synthesis, fermentation or other biotechnology-derived and enriched xanthophyll sources. A suitable form of lutein useful in the present invention is available commercially as e.g. Floraglo® Lutein (powder with lutein at 1%), or commercially available lutein in an oily form. The herein defined amounts of lutein refer to free lutein (i.e., equivalent to 100% pure lutein).

In a preferred embodiment, the composition comprises lutein in an amount ≥60 μg per 100 g of composition, preferably 60-430 μg per 100 g of composition, more preferably 70-400 μg per 100 g of composition, even more preferably 80-350 μg per 100 g of composition. Preferably, the composition is a powder composition and the weight is expressed per 100 g of powder composition. Expressed differently, the composition comprises lutein in an amount of 5-65 μg/100 ml of the composition, preferably 7-60 μg/100 ml of the composition, more preferably 8-55 μg/100 ml of the composition, even more preferably 8.5-50 μg/100 ml of the composition. The amounts expressed per 100 ml of the composition refer to ready-to-drink nutritional composition in liquid form, for example, after reconstituting the powder composition in water.

According to one preferred embodiment, the composition is an infant formula. Preferably, the infant formula according to the invention comprises lutein in an amount of 60-430 μg per 100 g of composition, preferably, 70-150 μg per 100 g of composition. Expressed differently, the infant formula comprises lutein in an amount of 5-65 μg/100 ml of the composition, preferably 6.5-60 μg/100 ml of the composition, more preferably 8-30 μg/100 ml of the composition. The amounts expressed per 100 ml of the infant formula refer to ready-to-drink infant formula in liquid form, for example, after reconstituting the powder in water.

According to another preferred embodiment, the composition is a follow-on formula. Preferably, the follow-on formula according to the invention comprises lutein in an amount of 60-430 μg per 100 g of powder composition, preferably, 120-390 μg per 100 g of powder composition, more preferably, 180-350 μg per 100 g of powder composition. Expressed differently, the follow-on formula comprises lutein in an amount of 5-65 μg/100 ml of the composition, preferably 15-55 μg/100 ml of the composition, more preferably 20-45 μg/100 ml of the composition. The amounts expressed per 100 ml of the follow-on formula refer to ready-to-drink follow-on formula in liquid form, for example, after reconstituting the powder composition in water.

According to yet another embodiment, the composition is a young child formula. Preferably, the young child formula according to the invention comprises lutein in an amount of 60-430 μg per 100 g of powder composition, preferably, 120-390 μg per 100 g of powder composition, more preferably, 180-350 μg per 100 g of powder composition. Expressed differently, the young child formula comprises lutein in an amount of 5-65 μg/100 ml of the composition, preferably 15-55 μg/100 ml of the composition, more preferably 20-45 μg/100 ml of the composition. The amounts expressed per 100 ml of the young child formula refer to ready-to-drink young child formula in liquid form, for example, after reconstituting the powder composition in water.

### Non-Digestible Carbohydrates

According to a preferred embodiment, the composition according to the invention comprises non-digestible carbohydrates.

As used herein, the term “non-digestible carbohydrate” refers to oligosaccharides which are not digested in the intestine by the action of acids or digestive enzymes present in the human upper digestive tract, e.g. small intestine and stomach, but reach the distal portions of the intestines, such as the colon, intact where they are fermented by the human intestinal microbiota. For example, sucrose, lactose, maltose and maltodextrins are considered digestible saccharides.

Microbial metabolites of non-digestible carbohydrates include short chain fatty acids (SCFA), for example acetate, propionate, butyrate, lactate, among others. The inventors have surprisingly found that the combination of lutein with the fermentation products of non-digestible carbohydrates (SCFA's) can synergistically inhibit mast cell degranulation thereby preventing or treating symptoms or disorders associated with mast cell activation.

Accordingly, in preferred embodiments, the composition according to the invention comprises non-digestible carbohydrates selected from group comprising prebiotic oligosaccharides, human milk oligosaccharides, and combinations thereof.

Preferably, the composition comprises 80 mg to 4 g non-digestible carbohydrates per 100 ml, more preferably 150 mg to 2 g per 100 ml, even more preferably 300 mg to 1 g non-digestible carbohydrates per 100 ml. Based on dry weight, the composition preferably comprises non-digestible carbohydrates in an amount of 0.25 wt. % to 25 wt. % non-digestible carbohydrates, more preferably 0.5 wt. % to 10 wt. %, even more preferably 1.5 wt. % to 7.5 wt. %, based on total composition.

### Prebiotic Oligosaccharides

The nutritional composition according to the invention preferably comprises prebiotic oligosaccharides.

Prebiotic oligosaccharides are non-digestible oligosaccharides. Preferred prebiotic oligosaccharides have a DP in the range of 2 to 250, more preferably 2 to 60, most preferably below 40. Advantageously and most preferred, the prebiotic oligosaccharides are water-soluble (according to the method disclosed in L. Prosky et al, J. Assoc. Anal. Chem 71:1017-1023, 1988).

Suitable prebiotic oligosaccharides are at least one, more preferably at least two, preferably at least three selected from the group consisting of fructo-oligosaccharides, galacto-oligosaccharides, xylo-oligosaccharides, arabino-oligosaccharides, arabinogalacto-oligosaccharides, gluco-oligosaccharides, chito-oligosaccharides, glucomanno-oligosaccharides, galactomanno-oligosaccharides, mannan-oligosaccharides, and uronic acid oligosaccharides. The group of fructo-oligosaccharides includes inulins, the group of galacto-oligosaccharides includes transgalacto-oligosaccharides or beta-galacto-oligosaccharides, the group of gluco-oligosaccharides includes cyclodextrins, gentio- and nigero-oligosaccharides and non-digestible polydextrose, the group of galactomanno-oligosaccharides includes partially hydrolyzed guar gum, and the group of uronic acid oligosaccharides includes pectin degradation products (e.g. prepared from apple pectin, beet pectin and/or citrus pectin).

Preferably, the composition comprises at least one prebiotic oligosaccharide, more preferably at least two prebiotic oligosaccharide. Preferably, the composition comprises prebiotic oligosaccharides selected from the list comprising: fructo-oligosaccharides, galacto-oligosaccharides, or mixtures thereof. More preferably, the composition comprises fructo-oligosaccharides and galacto-oligosaccharides at a weight ratio between (20 to 2):1, more preferably (20 to 2):1, even more preferably (20 to 2):1, even more preferably (12 to 7):1. Most preferably the weight ratio is about 9:1.

The galacto-oligosaccharides preferably are beta-galacto-oligosaccharides. In a particularly preferred embodiment the present composition comprises beta-galacto-oligosaccharides ([galactose]n-glucose; wherein n is an integer ranging from 2 to 60, i.e. 2, 3, 4, 5, 6, . . . , 59, 60; preferably n is selected from 2, 3, 4, 5, 6, 7, 8, 9, and 10), wherein the galactose units are in majority linked together via a beta linkage.

Beta-galacto-oligosaccharides are also referred to as trans-galacto-oligosaccharides (TOS). Beta-galacto-oligosaccharides are for example sold under the trademark Vivinal™ (Borculo Domo Ingredients, Netherlands). Another suitable source is Bi2Munno (Classado). Preferably the TOS comprises at least 80% beta-1,4 and beta-1,6 linkages based on total linkages, more preferably at least 90%.

Fructo-oligosaccharide is a prebiotic oligosaccharide comprising a chain of beta-linked fructose units with a DP or average DP of 2 to 250, more preferably 2 to 100, even more preferably 10 to 60. Fructo-oligosaccharide includes inulin, levan and/or a mixed type of polyfructan. An especially preferred fructo-oligosaccharide is inulin. Fructo-oligosaccharide suitable for use in the compositions is also commercially available, e.g. Raftiline®HP (Orafti). Preferably the fructo-oligosaccharide has an average DP above 20.

In one preferred embodiment, the composition according to the invention comprises prebiotic oligosaccharides only, i.e., without human milk oligosaccharides.

Preferably, the composition comprises 80 mg to 4 g prebiotic oligosaccharides per 100 ml, more preferably 150 mg to 2 g per 100 ml, even more preferably 300 mg to 1 g prebiotic oligosaccharides per 100 ml. Based on dry weight, the composition preferably comprises prebiotic oligosaccharides in an amount of 0.25 wt. % to 25 wt. % prebiotic oligosaccharides, more preferably 0.5 wt. % to 10 wt. %, even more preferably 1.5 wt. % to 7.5 wt. %, based on total composition. Expressed per 100 g of the composition, prebiotics oligosaccharides are present in an amount of at least 3 g per 100 g composition, more preferably 3.5-8 g per 100 g composition. Typically, the composition is in powder form, for instance, an infant formula, follow-on formula and/or young child formula. More preferably, the prebiotic oligosaccharides are selected from galacto-oligosaccharides, fructo-oligosaccharides or a combination thereof.

### Human Milk Oligosaccharides

According to one embodiment, the nutritional composition according to the invention comprises human milk oligosaccharides.

“Human milk oligosaccharides” (HMOs) are present in human milk and are non-digestible carbohydrates built from the following monomers: D-glucose, D-galactose, N-acetylglucosamine, L-fucose and sialic acid (N-acetylneuraminic acid).

Preferably, the composition of the invention comprises human milk oligosaccharides selected from the group comprising, but not limited to, sialyloligosaccharides, such as 3-sialyllactose (3-SL), 6-sialyllactose (6-SL), lactosialyltetrasaccharide a,b,c (LST), disialyllactoNtetraose (DSLNT), sialyl-lactoNhexaose (S-LNH), DS-LNH, and fucooligosaccharide, such as (un) sulphated fucoidan oligosaccharide, 2′-fucosyllactose (2′-FL), 3-fucosyllactose e (3-FL), difucosyllactose, lacto-N-fucopenatose, (LNFP) I, II, III, IV Lacto-N-neofucopenaose (LNnFP), Lacto-N-difucosyl-hexaose (LNDH), and mixtures thereof.

In one preferred embodiment, the composition according to the invention comprises human milk oligosaccharides only, i.e., without prebiotic oligosaccharides.

In one preferred embodiment, the composition according to the invention comprises human milk oligosaccharides only, i.e., without prebiotic oligosaccharides. Based on dry weight, the present nutritional composition preferably comprises 0.038 wt. % to 12 wt. % HMOs, preferably 0.075 wt. % to 9 wt. % HMOs, more preferably 0.15 wt. % to 6 wt. % HMOs, even more preferably 0.3 wt. % to 2.5 wt. % HMOs. Expressed differently, the composition comprises human milk oligosaccharides in an amount of 0.5 mg to 5 g per 100 ml of the composition, preferably 1.0 mg to 4.5 g per 100 ml of the composition, more preferably 0.5 g to 4.0 g per 100 ml of the composition, even more preferably 1.0 g to 3.5 g per 100 ml of the composition, most preferably 1.5 g to 3.0 g/100 ml of the composition. The amounts expressed per ml of the composition refer to ready-to-drink nutritional composition in liquid form. Based on energy, the present nutritional composition preferably comprises 0.008 to 2.5 g HMOs per 100 kcal, preferably 0.015 to 2.5 g HMOs per 100 kcal, more preferably 0.03 to 1.0 g HMOs per 100 kcal, even more preferably 0.06 to 0.5 g HMOs per 100 kcal. A too high amount will result in an increase the risk of osmotic diarrhea, which will counteract the beneficial effects of the mix.

Preferably, human milk oligosaccharides are selected from 2′-fucosyllactose (2′-FL), 3-fucosyllactose (3-FL), 3-sialyllactose (3-SL), 6-sialyllactose (6-SL), lacto-N-tetrose (LNT), lacto-N-neotetrose (LNnT), or combinations thereof. More preferably, the composition comprises 2′-FL.

2′-FL, preferably α-L-Fuc-(1→2)-β-D-Gal-(1→4)-D-Glc, is commercially available for instance from Sigma-Aldrich. Alternatively, it can be isolated from human milk, for example as described in Andersson & Donald, 1981, J Chromatogr. 211:170-1744, or produced by genetically modified micro-organisms, for example as described in Albermann et al, 2001, Carbohydrate Res. 334:97-103.

The nutritional composition of the present invention preferably comprises at least one human milk oligosaccharide selected from the group consisting of 2′-FL, 3-FL, 3′-SL and 6′-SL.

In a preferred embodiment, a nutritional composition according to the invention comprises at least 0.005 g of the sum of 2′-FL, 3-FL, 3′-SL and 6′-SL per 100 ml, more preferably at least 0.01 g, more preferably at least 0.02 g, even more preferably at least 0.04 g of the sum of 2′-FL, 3-FL, 3′-SL and 6′-SL per 100 ml. Based on dry weight, the present nutritional composition preferably comprises at least 0.038 wt. % of the sum of 5 2′-FL, 3-FL, 3′-SL and 6′-SL, more preferably at least 0.075 wt. %, more preferably at least 0.15 wt. % of the sum of 2′-FL, 3-FL, 3′-SL and 6′-SL, even more preferably at least 0.3 wt. %. Based on energy, the present nutritional composition preferably comprises at least 0.008 g of the sum of 2′-FL, 3-FL, 3′-SL and 6′-SL per 100 kcal, more preferably at least 0.015 g per 100 kcal, more preferably at least 0.03 g per 100 kcal, even more preferably at least 0.06 per 100 kcal.

Preferably the nutritional composition according to the invention comprises as a HMOS essentially 2′-FL, that means at least 95 wt. % of the HMOS consists of 2′-FL. Preferably, a nutritional composition according to the invention comprises 0.01 g to 1 g 2′-FL per 100 ml, more preferably 0.02 g to 0.5 g, even more preferably 0.04 g to 0.2 g 2′-FL per 100 ml. Based on dry weight, the present nutritional composition preferably comprises 0.075 wt. % to 8 wt. % 2′-FL, more preferably 0.15 wt. % to 4 wt. % 2′-FL, even more preferably 0.3 wt. % to 1.5 wt. % 2′-FL. Based on energy, the present nutritional composition preferably comprises 0.015 to 1.5 g 2′-FL per 100 kcal, more preferably 0.03 to 0.75 g 2′-FL per 100 kcal, even more preferably 0.06 to 0.4 g 2′-FL per 100 kcal.

In yet another preferred embodiment, a mixture of prebiotic oligosaccharides and human milk oligosaccharides is present. Preferably, the composition comprises at least two different non-digestible carbohydrates wherein at least two non-digestible carbohydrates are selected from either the group of prebiotics oligosaccharides or from the group of human milk oligosaccharides.

According to another embodiment, the nutritional composition comprises non-digestible carbohydrates, preferably at least two different non-digestible carbohydrates, more preferably, two different sources of non-digestible carbohydrates. Preferably, the at least two different non-digestible carbohydrates include a non-digestible carbohydrate selected from the group of prebiotics oligosaccharides and a non-digestible carbohydrate selected from the group of human milk oligosaccharides. More preferably, the composition comprises galacto-oligosaccharide and fructo-oligosaccharide in combination with 2′-FL and/or LNT, preferably 2′-FL.

Preferably, the weight ratio of human milk oligosaccharides (for instance FL, preferably 2′-FL) to prebiotic oligosaccharide (preferably, galacto-oligosaccharide) is from 5 to 0.05, more preferably 5 to 0.1, more preferably from 2 to 0.1. Preferably the weight ratio human milk oligosaccharides (for instance FL, preferably 2′-FL) to prebiotic oligosaccharide (preferably, fructo-oligosaccharide, more preferably inulin) is from 10 to 0.05, more preferably 10 to 0.1, more preferably from 2 to 0.5.

### Nutritional Compositions and Other Components

The present composition is preferably enterally administered, more preferably orally. The composition of the present invention includes dry food, preferably a powder, which is accompanied with instructions as to admix said dry food mixture with a suitable liquid, preferably with water.

In one embodiment, the nutritional composition is a powder. Suitably, the nutritional composition is in a powdered form, which can be reconstituted with water or other food grade aqueous liquid, to form a ready-to drink liquid, or is in a liquid concentrate form that should be diluted with water to a ready-to-drink liquid.

The present composition preferably comprises a lipid component, protein component, carbohydrate component and combinations thereof.

Preferably, the nutritional composition according to the invention is a nutritionally complete composition, that is, the composition comprises lipids, carbohydrates, and proteins.

According to a preferred embodiment, the human is an infant or young child. Preferably, the infant or young child is between 0-60 months of age, more preferably 0-36 months of age, even more preferably 6-24 months of age, most preferably 6-18 months of age.

Accordingly, preferred nutritional compositions for infants or young children include infant formula, follow-on formula, young child formula/growing-up milk, milk fortifiers, nutritional supplements, etc. Preferably, the nutritional composition is an infant formula, follow-on formula, or young child formula/growing-up milk. Preferably, the compositions are in the form of powders for reconstitution in a liquid prior to consumption. The present composition can be advantageously applied as a complete nutrition for infants. The compositions are synthetic compositions, i.e., are not or does not comprise human breast milk.

In the present invention, infant formula refers to nutritional compositions, artificially made, intended for infants of 0 to about 4 to 6 months of age and are intended as a substitute for human milk. Typically, infant formulae are suitable to be used as sole source of nutrition. Such infant formulae are also known as starter formula. Follow-on formula for infants starting with at 4 to 6 months of life to 12 months of life are intended to be supplementary feedings for infants that start weaning on other foods. Infant formulae and follow-on formulae are subject to strict regulations, for example for the EU regulations no. 609/2013 and no. 2016/127. In the present context, young child formula refers to nutritional compositions, artificially made, intended for infants of 12 months to 36 months, which are intended to be supplementary feedings for infants. In the context of the present invention, young child formula can also be named growing-up milk.

Nutritional compositions for children are preferably for children between 3 to 12 years of age, preferably 2 to 10 years of age, more preferably 3 to 6 years of age. Non-limiting examples of compositions for children include nutritional supplements, e.g. powders to be dispersed in a liquid such as water, milk or yoghurt, or ready-to-drink beverages.

The nutritional composition is preferably an infant formula or a follow-on formula or young child formula. In one embodiment, the nutritional composition is preferably an infant formula or follow-on formula or young child formula and preferably comprises 3 to 7 g lipid/100 kcal, preferably 4 to 6 g lipid/100 kcal, more preferably 4.5 to 5.5 g lipid/100 kcal, preferably comprises 1.7 to 3.5 g protein/100 kcal, more preferably 1.8 to 3.0 g protein/100 kcal, more preferably 1.8 to 2.5 g protein/100 kcal and preferably comprises 5 to 20 g digestible carbohydrate/100 kcal, preferably 6 to 16 g digestible carbohydrate/100 kcal, more preferably 10 to 15 g digestible carbohydrate/100 kcal.

The nutritional composition preferably has an energy density of 60 kcal to 75 kcal/100 ml, more preferably 60 to 70 kcal/100 ml, when in a ready-to-drink form.

Preferably, the composition comprises 5-15 g proteins per 100 g composition, more preferably 8-14 g proteins per 100 g composition. Normally the composition is a powder, therefore per 100 g powder composition. Expressed differently, the composition comprises 0.8-2.5 g proteins per 100 ml, more preferably 1.0-2.0 g proteins per 100 ml of the ready-to-drink composition. The protein is intact, partially or fully hydrolyzed.

Lipids are preferably present in an amount between 15-35 g per 100 g composition, preferably 20-30 g per 100 g composition. Expressed differently, the composition comprises 2.5-5 g lipids per 100 ml of the ready-to-drink composition, preferably 3.0-4.5 g lipids per 100 ml of the ready-to-drink composition.

Preferably, lipids include linoleic acid and alpha-linolenic acid. Preferably, the composition comprises 1.5-6 g linoleic acid per 100 g composition, preferably a powder composition. In another preferred embodiment, the composition comprises 150-550 mg alpha-linoleic acid per 100 g composition, preferably a powder composition.

Preferably the composition comprises DHA and/or ARA. Preferably, the composition comprises 65-150 mg DHA per 100 g composition, preferably a powder composition. Also preferably is a composition comprising from 100-200 mg ARA per 100 g composition, preferably a powder composition.

Preferably, the composition comprises digestible carbohydrates. Preferred digestible carbohydrate sources are lactose, glucose, sucrose, fructose, galactose, maltose, starch and maltodextrin. Preferably, the composition comprises at least 40 g digestible carbohydrates per 100 g of composition, preferably powder composition, more preferably 45-70 g digestible carbohydrates per 100 g composition. Per 100 ml, the composition comprises 5-9 g digestible carbohydrates, preferably 6-8 g digestible carbohydrates. The amounts expressed per 100 ml of the composition refer to ready-to-drink nutritional composition in liquid form, for example, after reconstituting the powder composition in water.

Lactose is the main digestible carbohydrate present in human milk. The nutritional composition preferably comprises lactose. The nutritional composition preferably comprises digestible carbohydrate, wherein at least 35 wt. %, more preferably at least 50 wt. %, more preferably at least 75 wt. %, even more preferably at least 90 wt. %, most preferably at least 95 wt. % of the digestible carbohydrate is lactose. Based on dry weight the nutritional composition preferably comprises at least 25 wt. % lactose, preferably at least 40 wt. % lactose.

According to one embodiment, the composition comprises casein phosphopeptides (CPP). Preferably, the composition comprises CPP in an amount of 5-10 mg per 100 ml of the composition, preferably 6.0-9.0 mg per 100 ml of composition. The amounts expressed per ml of the composition refer to ready-to-drink nutritional composition in liquid form. Per 100 g of the composition, preferably a powder composition, CPP is present in an amount ranging from 25-80 mg, preferably 40-70 mg. Preferably, the weight ratio of lutein to CPP ranges from 1:150 to 1:1000, preferably from 1:200 to 1:900.

In yet another embodiment, the composition comprises 1,3-Dioleoyl-2-palmitoyl glycerol (OPO). As used herein, 1,3-dioleoyl-2-palmitoylglycerol refers to fats commercially available, for example, as Betapol® from Loders Croklaan BV, Wormerveer, The Netherlands such as Betapol® B-55. Preferably, the composition comprises 2-8 g OPO per 100 g composition, preferably 3.5-6.5 g OPO per 100 g composition, preferably a powder composition. Expressed differently, the composition comprises 0.25-1.5 g OPO per 100 ml of the composition, preferably 0.4-1.0 g OPO per 100 ml of the composition, preferably the reconstituted, ready-to-drink composition.

Accordingly, according to another aspect the invention relates to a nutritional composition comprising carbohydrates, proteins and lipids, wherein the composition is a powder composition comprising, per 100 g:
a. an energy content of at least 400 kcal;
b. 60-430 μg lutein;
c. 3.5-8 g non-digestible carbohydrates comprising fructo-oligosaccharides and galacto-oligosaccharides;
d. 45-70 g digestible carbohydrates, preferably lactose;
the composition further comprising at least one of:
e. 25-80 mg casein phosphopeptides;
f. 2-8 g 1,3-Dioleoyl-2-palmitoyl glycerol.

Preferably, the nutritional composition comprises casein phosphopeptides.

The compositions according to the invention can also be used as a supplement for adults. Preferably the adults suffer or are at risk of suffering from mast cell degranulation disorders, for instance, individuals suffering from IgE-mediated and non-IgE-mediated mast cell degranulation disorders, individuals with higher prevalence of inflammatory disease such as cancer, diabetes, arthritis, irritable bowel syndrome (IBS), elderly (such as aging adults, particularly immune senescence in aging adults) and non-communicable disease (NCDs) in general. Preferably, the adults are suffer or are at risk of suffering from IgE-mediated and non-IgE-mediated mast cell degranulation disorders.

More preferably, the human subject is an adult, more preferably an adult aged above 50 years old, even more preferably above 55 years old. Compositions for human adults comprise nutritional supplements, e.g. powders to be dispersed in a liquid such as water, milk or yoghurt, ready-to-drink beverages, nutritionally complete enteral compositions, such as those suitable for tube feeding, capsules, tablets, and the like.

The present invention is illustrated by the following non-limiting examples.

### EXAMPLES

### Example 1

Using the β-hexosaminidase release assay, also known for short as the RBL-assay, IgE-dependent degranulation of mast cells was used to study the effect of lutein compositions.

Confluent growing RBL-2H3 cells (1×10⁵/well) in 96-wells flat bottom white culture plate were sensitized by 100 ng/ml mouse IgE α-DNP in 100 μl culture medium. After 18 hr of culture at 37° C., 5% CO₂, the cells were washed with 100 μl Tyrode's buffer (130 mM NaCl, 190 mM KCl, 1.4 mM CaCl₂), 1 mM MgCl₂, 5.6 mM glucose, 10 mM HEPES and 0.1% BSA in distilled water, pH 7.4). 25 μl of the compounds and 25 μl DNP-BSA 100 ng/ml to induce cross-linking were supplemented to the adhered cells. Cells were incubated for 1 hr at 37° C./5% CO₂. 50 μl 4-methyl umbelliferyl-N-acetyl-β-D-glucosaminide (4-MUG) 60.6 μg/ml in 5 ml citrate buffer (0.1 M citric acid in demi, pH 4.5) was added to the wells and incubated for 1 hr at 37° C./5% CO₂. The reaction was stopped by adding 100 μl glycinbuffer (15 g glycin and 11.7 g NaCl in 1 L demi, pH 10.7). Fluorescence, I_{excitation} 360 nm and Iₑₘᵢₛₛᵢₒₙ 460 nm was measured on Fluostar. Maximum content of beta-hexosaminidase was measured using 10 μl 1% v/v Triton X-100 in Tyrode's buffer. The maximum of IgE-dependent degranulation of RBL cells (positive control) was set to 100% and the confidence threshold for a relevant inhibitory effect was commonly set to 80%, meaning that all inhibition below that can be defined as inhibition of degranulation. Viability of cells was tested using WST-1-assay and showed no change in viability (100% viable, data not shown).

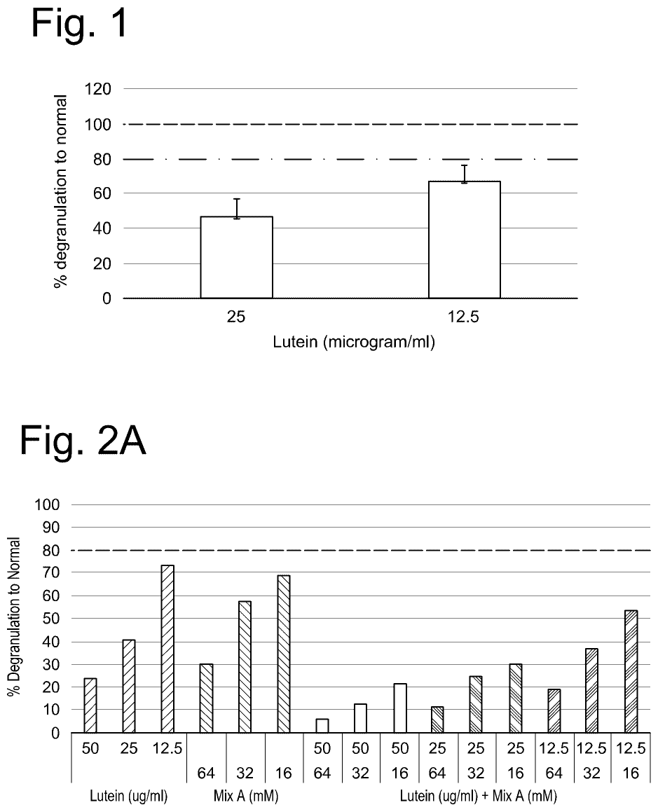

Lutein was tested at different concentrations respectively at a concentration of 25 and 12.5 μg/ml. The results are shown in FIG. 1.

### Example 2

A further RBL-assay (as described in Example 1) was performed based on the combination of lutein with short chain fatty acids (SCFA) acetate/propionate/butyrate which represent the microbial metabolites of non-digestible oligosaccharides.

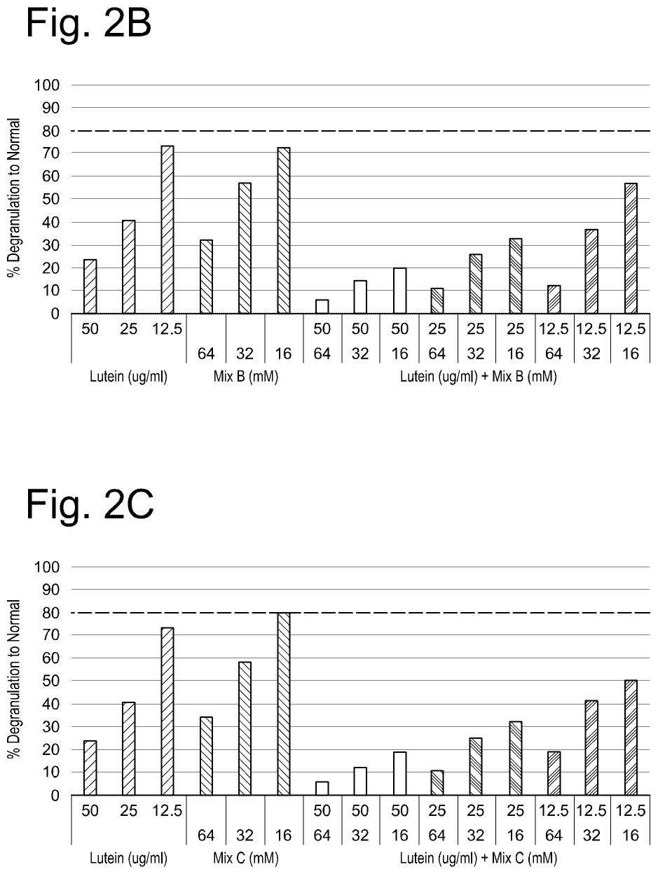

Tested concentrations of lutein were 50 μg/ml, 25 μg/ml and 12.5 μg/ml. Tested concentrations of the SCFA mixture were 64 mM, 32 mM and 16 mM and different ratios of acetate/propionate/butyrate were compared. The ratios of acetate to propionate to butyrate in the SCFA mixtures were respectively 75:20:5 (FIG. 2A), 85:11:4 (FIG. 2B) and 90:9:1 (FIG. 2C). The amounts of SCFAs in the mixtures is shown in Table A. The confidence threshold for a relevant inhibitory effect was set at 80%.

TABLE A

| SCFA | | Acetate | Propionate | Butyrate |
|---|---|---|---|---|
| mix | Ratio | mg/ml | mg/ml | mg/ml |
| A | 75/20/5 | 9.84 | 3.07 | 0.88 |
| B | 85/11/4 | 11.16 | 1.69 | 0.70 |
| C | 90/9/1 | 11.81 | 1.38 | 0.18 |

The results are shown in FIG. 2A-C. Surprisingly, the combination of lutein at all concentrations with different amounts (at different ratios) of SCFA synergistically inhibited mast cell degranulation.

### Example 3—Effect of Lutein on Non-IgE-Mediated Mast Cell Degranulation

The RBL-assay as described in Example 1 was repeated replacing sensitization with mouse IgE α-DNP and degranulation with DNP-BSA by compound 48/80 to study the effect of lutein on non-IgE-dependent degranulation of mast cells.

Compound 48/80 is a mixed polymer of p-methoxy-N-methyl phenylethylamine crosslinked by formaldehyde and widely used for non-IgE dependent stimulation of mast cells. The concentration of compound 48/80 used was 62.5 μg/ml. The maximum of non-IgE-dependent degranulation of RBL cells (positive control) was set to 100% and the confidence threshold for a relevant inhibitory effect was commonly set to 80%, meaning that all inhibition below that can be defined as inhibition of degranulation. Viability of cells was tested using WST-1-assay and showed no change in viability (100% viable, data not shown).

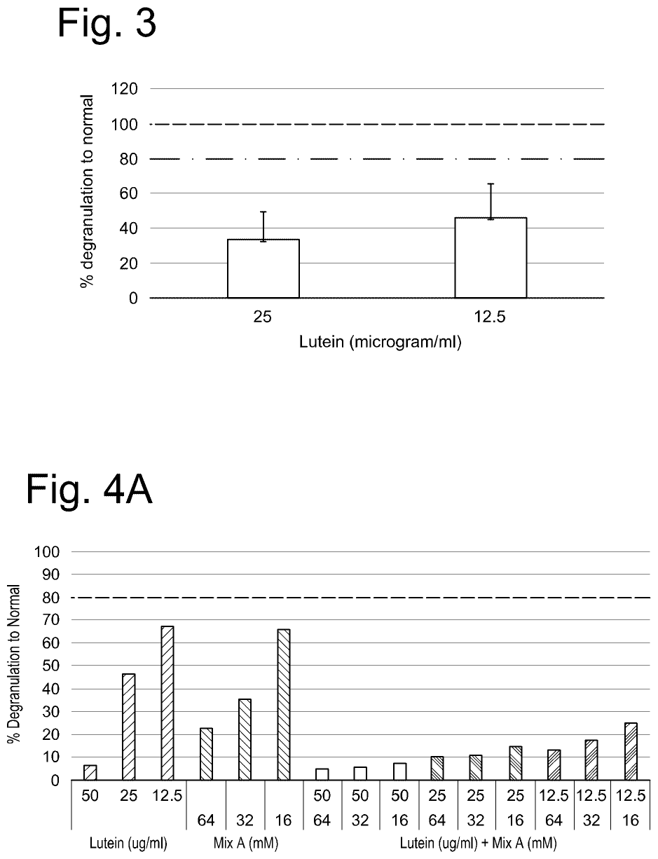

Lutein was tested at different concentrations respectively at a concentration of 25 and 12.5 μg/ml. The results are shown in FIG. 3.

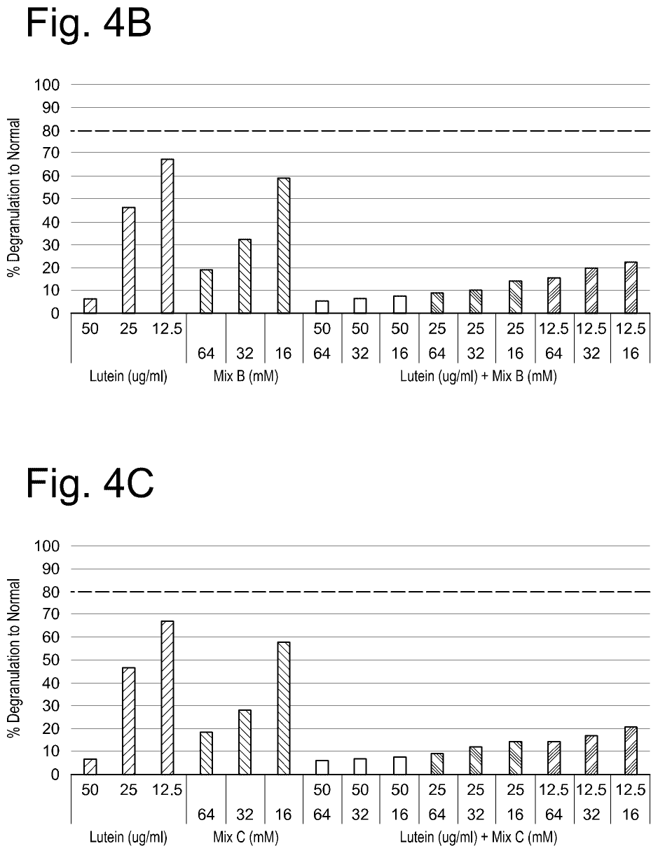

Further tested were combinations of lutein with SCFA mixtures as described in Example 2 and table A. The results are shown in FIGS. 4(a)-(c).

1.-17. (canceled)

18. A method for the prevention or treatment of a mast cell degranulation associated disorder in a human subject in need thereof.

19. The method according to claim 18, wherein the disorder is allergy.

20. The method according to claim 19, wherein the allergy is a food allergy, such as milk and/or egg allergy.

21. The method according to claim 18, wherein the treatment or prevention inhibits allergic symptoms in a human subject, wherein the allergic symptoms are caused by mast cell degranulation.

22. The method according to claim 18, wherein the composition comprises ≥60 μg lutein per 100 g of composition,

23. The method according to claim 22, wherein the composition comprises 60-430 μg lutein per 100 g of composition.

24. The method according to claim 18, wherein the composition comprises non-digestible carbohydrates.

25. The method according to claim 18, wherein the composition comprises prebiotic oligosaccharides, preferably selected from fructo-oligosaccharides (FOS), galacto-oligosaccharides (GOS), or mixtures thereof.

26. The method according to claim 18, wherein the composition comprises human milk oligosaccharides.

27. The method according to claim 26, wherein the human milk oligosaccharide is 2′-fucosyllactose (2′-FL).

28. The method according to claim 18, wherein the composition comprises non-digestible carbohydrates in an amount ranging between 0.25 wt. % to 25 wt. %, based on dry weight of the composition.

29. The method according to claim 18, wherein the composition comprises lactose.

30. The method according to claim 18, wherein the human subject is an infant or young child.

31. The method according to claim 18, wherein the composition is an infant formula, follow-on formula or young child formula.

32. The method according to claim 18, wherein the human subject is an aging adult.

33. A nutritional composition comprising carbohydrates, proteins and lipids, wherein the composition is a powder composition comprising, per 100 g:
a. an energy content of at least 400 kcal;
b. 60-430 μg lutein;
c. 3.5-8 g non-digestible carbohydrates comprising fructo-oligosaccharides and galacto-oligosaccharides;
d. 45-70 g digestible carbohydrates, preferably lactose;
the composition further comprising at least one of:
e. 25-80 mg casein phosphopeptides; or
f. 2-8 g 1,3-Dioleoyl-2-palmitoyl glycerol.

34. The nutritional composition of claim 33, wherein the composition comprises casein phosphopeptides.
